(19) United States
(12) Patent Application Publication
(10) Pub. No.: US 2011/0275962 A1
(43) Pub. Date: Nov. 10, 2011

(54) ACOUSTICAL SWITCH AND CATHETER COMPRISING ACOUSTICAL SWITCH

(75) Inventors: Szabolcs Deladi, Eindhoven (NL); Jan F. Suijver, Eindhoven (NL); Godefridus Antonius Harks, Eindhoven (NL); Johannes Wilhelmus Weekamp, Eindhoven (NL); Antonius J.J. Rademakers, Eindhoven (NL); Maya Ella Barley, Eindhoven (NL)

(73) Assignee: KONINKLIJKE PHILIPS ELECTRONICS N.V., EINDHOVEN (NL)

(21) Appl. No.: 13/127,089

(22) PCT Filed: Nov. 5, 2009
(86) PCT No.: PCT/IB09/54907

§ 371 Date: Jul. 27, 2011

(30) Foreign Application Priority Data
Nov. 12, 2008 (EP) 08168895.4

(51) Int. Cl. A61N 7/00

(52) U.S. Cl. 601/2

(57) ABSTRACT

The invention relates to an acoustical switch wherein the idea to the ultrasound propagation direction may be changes without moving the switch. The switch device comprises two sheets of acoustically transparent material. The sheets constitute opposite walls of a housing. The switch device further comprises one or more orifices for allowing conduction of fluid into and/or out of said housing. The switch device may be made reflective by filling the housing with a gas via the one or more orifices. Moreover, the switch device may be made transmissive to ultrasound by filling the housing with a liquid via the one or more orifices and/or by subjecting the housing to underpressure via the one or more orifices. The acoustical switch device fits well within a catheter, which has severe dimensional limitations.

### FIELD OF THE INVENTION

The present invention relates to an acoustical switch device for ultrasound and a method of controlling the state of an acoustical switch device for ultrasound. The invention moreover relates to an ultrasound catheter and a method for directing the ultrasound from an ultrasound catheter. Moreover, the invention relates to a radio frequency ablation catheter comprising an ultrasound transducer and an acoustical switch device.

### BACKGROUND OF THE INVENTION

Various medical methods for monitoring and/or treating a patient comprises inserting a catheter into a body cavity, duct or vessel for allowing e.g. drainage or injection of fluids, radiation, ablation or access by surgical instruments.

A requirement for catheters is adequate control and/or monitoring. The spatial orientation of the catheter with respect to the tissue to be monitored may change from perpendicular to parallel. Therefore it is essential that the monitoring can switch according to the orientation of the catheter.

One example of a catheter used in a minimally-invasive treatment of cardiac arrhythmias, is the Radio Frequency (RF) Ablation Catheter, which aims at electrically inactivating cardiac tissue through local heating by means of RF energy. However, RF catheter ablation procedures still have a significant drawback with regard to the difficulty of actively controlling the ablation settings during treatment. Currently, the therapist relies on his/her own expertise to determine the optimal parameters for ablation, such as power, temperature, and duration. However, these settings typically vary significantly due to sizable intra-patient differences of thickness of the local heart wall, perfusion, blood pressure and velocity, heart rhythm, etc. Although a highly-skilled therapist is able to achieve successes with this approach, it is not always the case, and there are serious consequences for the patient when an error is made.

The two major therapy-related problems result from either the under-heating or the overheating of the site. In the case of under-heating, the tissue is not sufficiently coagulated to form the arrhythmia-blocking lesion desired by the therapist. This can lead to persistent or recurring symptoms in the patient, and the requirement for subsequent treatment(s), longer periods of hospitalisation, and greater risks of stroke and embolism. The other extreme, overheating, either causes rupturing of the tissue at the treatment site, releasing potentially life-threatening particles into the blood stream, or causes damage to neighbouring organs and tissues. In the case that other organs are affected, fistulas can develop and these are often life-threatening.

A requirement for RF catheters is more adequate control. A device providing feedback of the lesion development in the tissue and information about the depth of the lesion with respect to the thickness of the tissue at the treatment site would prevent injury and death from under-heating and overheating in RF catheter procedures. In most of the ablation procedures the spatial orientation of the catheter with respect to the tissue changes from perpendicular to parallel, therefore it is essential that the ultrasonic lesion monitoring can switch according to the procedure.

The document U.S. Pat. No. 5,546,360 describes a composite acoustic lens able to be steered internally through electrical control. The lens is steerable by virtue of its material: it is electrorheological. Its bulk modulus, and the resulting speed of sound, can be changed electrically. Controlling the gradient of the index of refraction allows the steering to be adjusted precisely, continuously, and quickly. However, the lens is to be used with an array of transducers, being relatively large and not readily applicable to a catheter. Furthermore, the change in direction of monitoring by means of this lens is limited because of the operation principle of the lens.

Hence, an improved device for monitoring would be advantageous, and in particular a small device capable of monitoring in different directions would be advantageous. Moreover, a catheter having monitoring capabilities in different directions would be advantageous.

### SUMMARY OF THE INVENTION

Accordingly, the invention preferably seeks to mitigate, alleviate or eliminate one or more of the above mentioned disadvantages singly or in any combination. In particular, it may be seen as an object of the present invention to provide an acoustical switching device that solves the above mentioned problems of the prior art with regard to monitoring in different directions.

This object and several other objects are obtained in a first aspect of the invention by providing an acoustical switch device for ultrasound, wherein said switch device comprises two sheets of acoustically transparent material, said sheets constituting opposite walls of a housing, said switch device further comprising one or more orifices for allowing conduction of fluid into and/or out of said housing.

The invention is particularly, but not exclusively, advantageous for obtaining a switch device that may be arranged for being transmissive or reflective in accordance with whether the housing is filled with gas or liquid or emptied. The two sheets forming opposite walls of the housing are separated by a gap, except when the housing is subject to underpressure. This gap may be filled with fluid, viz. a gas or a liquid, the choice of which influences the transmission properties of the ultrasound switch. Since the switch device thus may provide a transmission or a reflection of ultrasound depending on the presence or absence of a fluid within the housing, it may be controlled to switch between transmission and reflection without changing the position of the switch device mechanically.

It should be noted that the opposite walls of the housing preferably are substantially parallel to each other. The sheets may for instance be of a plastic material, e.g. TPX foil, having a very low ultrasound reflection due to good acoustical impedance match with water. The term underpressure is meant to denote any appropriately partial vacuum or low pressure sufficiently low to make the walls of the housing come together.

According to another aspect of the invention, the acoustical switch device is made reflective by filling the housing with a gas via the one or more orifices. Such a gas may be air or any other appropriate gas. When the housing of the switch device is filled with one or more gasses, the acoustical impedance mismatch of the device will be large, resulting in a reflection of almost all the ultrasound. By orienting the switch device in various directions, the ultrasound may be redirected. If the acoustical mismatch and the angle between the ultrasound beam and the acoustical switch are sufficiently large, total internal reflection of the ultrasound can occur, whereby 100% of the ultrasound beam is reflected by the acoustical switch.

According to another aspect of the invention, the switch device is made transmissive to ultrasound by filling the housing with a liquid via the one or more orifices. When the device is filled with liquid that has the same acoustical properties as the environment in which the device is immersed, the switch device will be transparent to ultrasound, which may propagate unhindered from an ultrasound transducer. When the switch device is used in water or blood, the liquid to be filled into the housing may e.g. be water.

Alternatively, the switch device may be made transmissive to ultrasound by subjecting the housing to underpressure via the one or more orifices. In this case the acoustically transparent, opposite sheets will come into contact with each other, therefore forming an acoustically transparent sheet having a thickness of the sum of the thicknesses of the two sheets. In this case the device will also be transparent to ultrasound, due to the acoustical transparency of the sheet material, and will allow the propagation of the ultrasound along the longitudinal axis of the transducer.

According to second aspect, the invention relates to a method of controlling the state of an acoustical switch device for ultrasound, where said switch device comprises two sheets of acoustically transparent material, said sheets constituting opposite walls of a housing, said method comprising the one or more of the following steps: via the one or more orifices, filling the housing with a gas in order to bring the acoustical switch into a reflective state, and/or via the one or more orifices, subjecting the housing to underpressure or the filling housing with a liquid in order to bring the acoustical switch into a transmissive state. The liquid to be filled into the housing in the liquid-filled alternative should have the same acoustical properties as the environment in which the device is immersed. If the device is used in blood, the liquid could advantageously be water.

According to a third aspect, the invention moreover relates to an ultrasound catheter comprising: an ultrasound transducer arranged for transmitting ultrasound; and a switch device according to the invention. Such a catheter has monitoring capabilities in at least two different directions due to the ability of the switch device to be transmissive or reflective depending on whether a fluid is present in the housing or not. Thus, the monitoring direction of the catheter may be changed without moving or changing the orientation of the catheter itself.

Preferably, the ultrasound catheter further comprises a catheter enclosure accommodating at least part of the switch device and further comprising a liquid, where one or more walls of the catheter enclosure comprising acoustically transparent material.

The ultrasound transducer of the ultrasound catheter may be a single transducer or an array of transducers, and the ultrasound transducer may comprise any appropriate transducer, such as a transducer of one or more of the following types: PZT, PVDF, cMUT, pMUT.

The transducer of the ultrasound catheter may be rotatable around an axis angled in relation to an axis of emission of ultrasound from the transducer. This would render it possible to obtain two dimensional ultrasound imaging. The rotational axis might be perpendicular to the axis of emission of ultrasound; for example the rotational axis might be perpendicular to the plan of the FIGS. 1 to 4.

The ultrasound transducer may further comprise a lens arranged for spreading the ultrasound emitted from the ultrasound transducer. Hereby, the scanning angle of the ultrasound catheter may be enlarged significantly so as to enable scanning ultrasound emitted from the ultrasound transducer over a certain sector. Advantageously, the lens is a liquid lens operable by the electro wetting principle.

The ultrasound from the ultrasound transducer of the ultrasound catheter may have an incident angle upon the switch device of about 45°. When the switch device is in its reflecting state, the reflected ultrasound beam is also expected to be 45°, therefore providing monitoring of an object perpendicular to the direction of ultrasound emitted from the transducer.

Advantageously, the ultrasound catheter further comprises means for actuating the switch device mechanically. Even with a limited mechanical actuation of the acoustical switch device, the ultrasound beam from the ultrasound transducer may be scanned along the throughout directions in the vicinity of the two monitoring directions of the ultrasound catheter.

According to a fourth aspect, the invention relates to a method for directing the ultrasound from an ultrasound catheter comprising an ultrasound transducer arranged for transmitting ultrasound and an acoustical switch device according to the invention, said method comprising: filling the housing with a gas in order to bring the acoustical switch into a reflective state or subjecting the housing to underpressure or the filling housing with a liquid in order to bring the acoustical switch into a transmissive state, and subjecting the switch device to ultrasound.

According to a fifth aspect, the invention relates to a radio frequency ablation catheter comprising an ultrasound catheter according to the invention. The ultrasound catheter provides the radio frequency ablation catheter with the ability to monitor ablation in at least two directions. This is especially advantageous in that orientation of the radio frequency ablation catheter with regard to the tissue may change from perpendicular to parallel for different ablation sites. Thus, it is advantageous to be able to monitor or image both in parallel and perpendicular to the catheter axis.

The different aspects of the present invention may each be combined with any of the other aspects. These and other aspects of the invention will be apparent from and elucidated with reference to the embodiments described hereinafter.

### BRIEF DESCRIPTION OF THE DRAWING

The present invention will now be explained, by way of example only, with reference to the accompanying Figures, where

FIG. 1 is a schematic drawing of an acoustical switch device according to the invention;

FIGS. 2 to 4 are schematic drawings of embodiments of ultrasound catheters according to the invention, and

FIG. 5 is a flow-chart of a method according to the invention.

Throughout the figures, like references denote like elements.

### DETAILED DESCRIPTION OF EMBODIMENTS

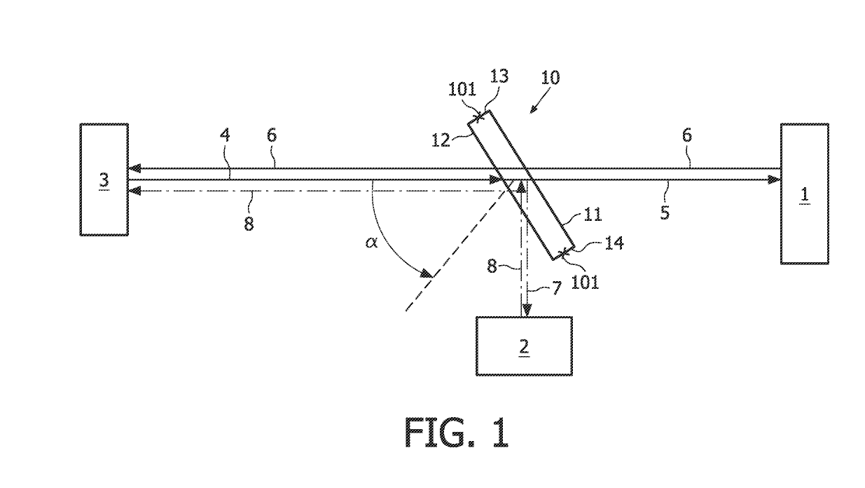

FIG. 1 is a schematic drawing of an acoustical switch device 10 according to the invention in a set-up with an ultrasound transducer 3 and objects 1 and 2 to be observed by means of ultrasound reflections.

The acoustical switch device 10 comprises a housing having two opposite walls 11 and 12 as well as end walls 13, 14. The housing of the switch device 10 moreover comprises one or more orifices 101 for allowing conduction of fluid into the housing and/or conduction of fluid out of the housing.

The opposite walls 11 and 12 of the housing as shown in FIG. 1 define a gap. This gap between the two opposite walls 11, 12 may be subjected to underpressure or filled with various fluids by a syringe connected, e.g. via a thin hollow pipe, to one of the one or more orifices of the switch device 10. The walls 11, 12 are of acoustically transparent materials, e.g. TPX foils which have very low ultrasound reflection due to the good acoustical impedance match with water. The walls 11, 12 of are typically parallel sheets and may have a thickness of 50 μm, separated by a gap of 0.5 mm.

In the case where housing of the switch device 10 is filled with a gas, it is made reflective to an ultrasound beam from the ultrasound transducer 3 with a reflection angle equal to the incidence angle.

The switch device 10 may be made transmissive to an ultrasound beam by filling the housing with a liquid via an orifice amongst the one or more orifices. The liquid may e.g. be water. Alternatively, the acoustical switch device 10 could be made transmissive to ultrasound by subjecting the housing to sufficient underpressure via the one or more orifices, so that the opposite walls 11 and 12 come into contact and join each other. In this case an ultrasound beam from an ultrasound transducer 3 will be transmitted through the switch device 10, in that the opposite walls 11 and 12 of the switch device 10 function as a single acoustically transparent material, having the thickness of the sum of the thicknesses of the walls 11 and 12.

The acoustical switch device 10 is placed between an ultrasound transducer 3 and an object 1 along of the ultrasound emitted from the transducer. An object 2 is placed right below the acoustical switch device 10, perpendicular to the longitudinal axis of the ultrasound emitted from transducer.

The acoustical switch device 10 is oriented such that an incident ultrasound beam 4 hit the face of the device at angle α; advantageously, a may be equal to 45°, in which case a reflected ultrasound beam would also be 45°, thereby rendering it possible to detect the object 2 when the switch device 10 is in its reflective state, viz. when the gap between the walls 11 and 12 of the switch device 10 is filled with gas.

When the switch device 10 is in its transmissive state, viz. either filled with an appropriate liquid or subject to vacuum or appropriate underpressure, the ultrasound beam 4 is transmitted through the switch device, viz. a transmitted ultrasound beam 5. The transmitted ultrasound beam 5 may be reflected by the object 1, into a reflected ultrasound beam 6 which passes through the switch device 10 being in its transmissive state and returns to the ultrasound transducer 3.

When the switch device 10 is in its reflective state, viz. filled with an appropriate gas, such as air, the ultrasound beam 4 is reflected by the switch device 10 to a reflected ultrasound beam 7. The reflected ultrasound beam 7 may be reflected by the object 2, into a reflected ultrasound beam 8 which is reflected by the switch device 10 being in its reflective state and returns to the ultrasound transducer 3.

Thus, depending on the state of the switch device 10, the object 1 or the object 2 may be observed by subjected it to ultrasound from the ultrasound transducer 3.

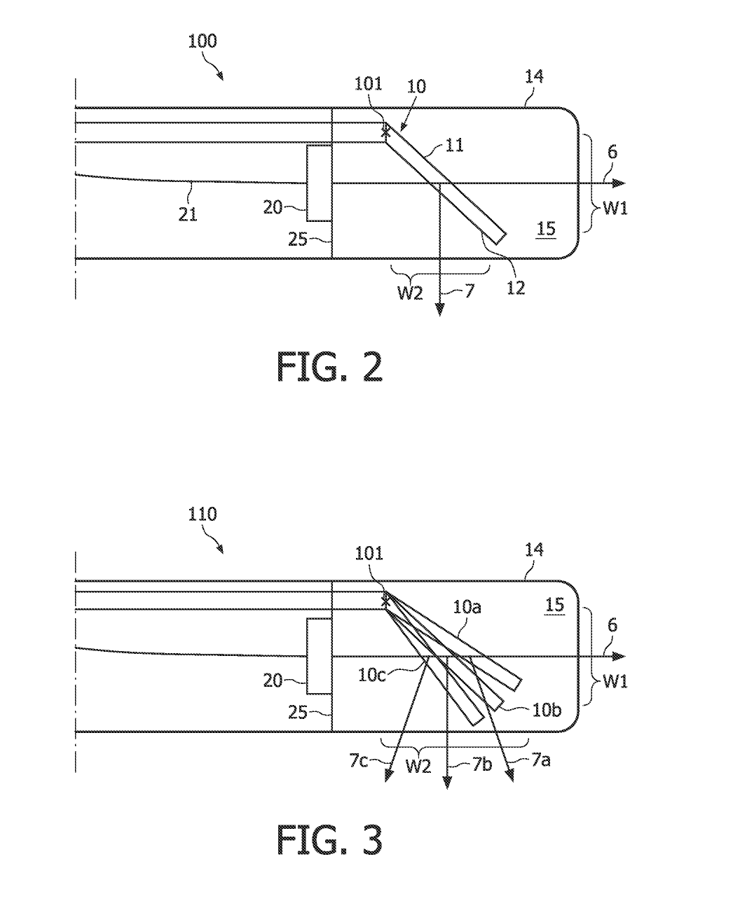

FIG. 2 is a schematic drawing of an embodiment of an ultrasound catheter 100 according to the invention.

The catheter 100 comprises an acoustic switch device 10 comprising a housing defined by the walls 11 and 12. The catheter 100 comprises an outer casing 14, which together with a wall 25 define an enclosure 15, wherein the housing of the switch device 10 is arranged. The catheter 100 moreover comprises an ultrasound transducer 20 powered via a lead 21. The casing 14 comprises two windows, W1 and W2, which are transmissive to ultrasound. Alternatively, all of the casing 14 might be of acoustical transmissive material. However, the portions of the casing not comprising the acoustical windows W1 and W2, could can be of other material, which may have electrically conductive segments in order to apply RF energy for tissue treatment. The enclosure 15 of the catheter tip may be filled with liquid, e.g. saline water.

The housing of the switch device 10 is arranged at an angle of about 45° along the longitudinal axis of the transducer, so that ultrasound emitted from the transducer 20 has an incidence angle of 45° on the housing of the switch device 10. In FIG. 2, a transmitted ultrasound beam is denoted by the numeral 6, in the case where the acoustical switch device 10 is transparent to ultrasound, i.e. filled with liquid or under vacuum, and a reflected ultrasound beam is denoted by the numeral 7, in the case where the acoustical switch device 10 is reflective to ultrasound, i.e. filled with a gas such as air.

The catheter 100 is thus a catheter where the field of view or the imaging field can be changed without moving the catheter mechanically. One example of an application of the catheter 100 is for the ablation of the left atrium for atrial fibrillation treatment, where an RF ablation catheter changes its spatial orientation with respect to the tissue during the procedure.

FIG. 3 is a schematic drawing of another embodiment of an ultrasound catheter 110 according to the invention.

The catheter 110 comprises an acoustic switch device which may be actuated mechanically to be placed within a range of positions, whereof three positions are denoted by the numerals 10a, 10b and 10c.

The catheter 110 comprises an outer casing 14, which together with a wall 25 define an enclosure 15, wherein the housing of the switch device is arranged. The catheter 110 moreover comprises an ultrasound transducer 20 powered via a lead 21. The casing 14 comprises two windows, W1 and W2, which are transmissive to ultrasound. Alternatively, all of the casing 14 might be of acoustical transmissive material. However, the portions of the casing not comprising the acoustical windows W1 and W2, could can be of other material, which may have electrically conductive segments in order to apply RF energy for tissue treatment. The enclosure 15 of the catheter tip may be filled with liquid, e.g. saline water.

The housing of the switch device in the position 10b is arranged at an angle of about 45° along the longitudinal axis of the transducer, so that ultrasound emitted from the transducer 20 has an incidence angle of 45° on the housing of the switch device 10. In FIG. 2, a transmitted ultrasound beam is denoted by the numeral 6, in the case where the acoustical switch device 10 is transparent to ultrasound, i.e. filled with liquid or under vacuum, and a reflected ultrasound beam is denoted by the numeral 7, in the case where the acoustical switch device 10 is reflective to ultrasound, i.e. filled with a gas such as air. In the positions 10a and 10c the incidence angle of the beam from the ultrasound transducer 20 to the housing of the switch device 10a, 10c are different from 45°, whereby the ultrasound beam may inpinge on a range of angles upon an object (not shown in FIG. 3) below the catheter as indicated by the arrows 7a, 7b and 7c corresponding to ultrasound beams transmitted from the housing of the switch device when in the positions 10a, 10b and 10c, respectively.

Thus, with limited mechanical actuation of the acoustical switch it is possible to scan the ultrasound beam along the acoustical window W2. It should be noted that the diameter of the active element of the transducer is limited by the range of positions in which the acoustical switch device can be operated.

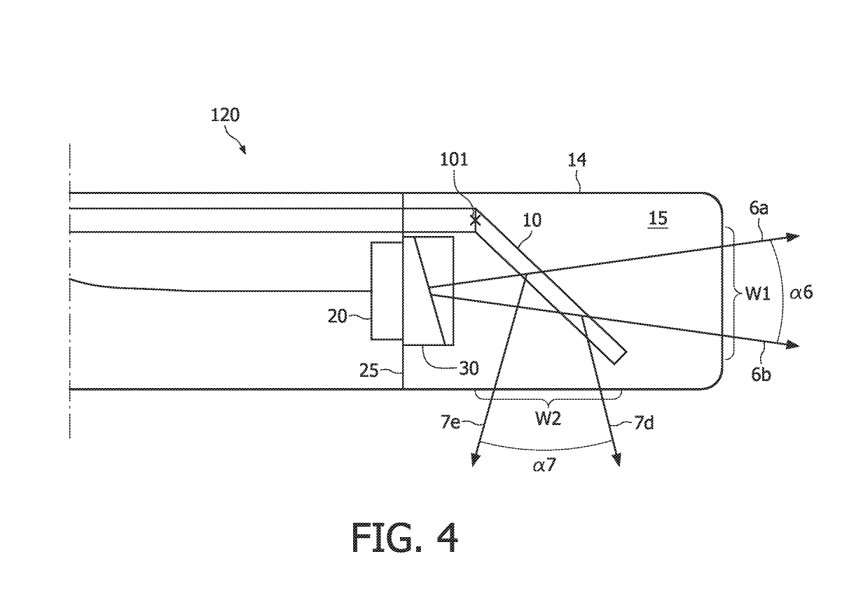

FIG. 4 is a schematic drawing of yet another embodiment of an ultrasound catheter 120 according to the invention.

The catheter 120 comprises an acoustic switch device 10 and an outer casing 14, which together with a wall 25 define an enclosure 15, wherein the housing of the switch device 10 is arranged. The catheter 120 moreover comprises an ultrasound transducer 20 powered via a lead 21. The casing 14 comprises two windows, W1 and W2, which are transmissive to ultrasound. Alternatively, all of the casing 14 might be of acoustical transmissive material. However, the portions of the casing not comprising the acoustical windows W1 and W2, could can be of other material, which may have electrically conductive segments in order to apply RF energy for tissue treatment. The enclosure 15 of the catheter tip may be filled with liquid, e.g. saline water.

Again, the switch device 10 may be made transmissive to an ultrasound beam by filling the housing with a liquid via an orifice amongst the one or more orifices. The liquid may e.g. be water. Alternatively, the acoustical switch device 10 could be made transmissive to ultrasound by subjecting the housing to sufficient underpressure via the one or more orifices, so that the opposite walls 11 and 12 come into contact and join each other. In this case an ultrasound beam from an ultrasound transducer 3 will be transmitted through the switch device 10, in that the opposite walls 11 and 12 of the switch device 10 function as a single acoustically transparent material, having the thickness of the sum of the thicknesses of the walls 11 and 12.

The catheter 120 moreover comprises a lens 30, e.g. a liquid lens, rendering it possible to sweep an ultrasound beam emitted from the ultrasound transducer 20 over a large area in forward and sideward/downwards configurations. The area in the forward direction, viz. the area between the transmitted ultrasound beams 6a and 6b, is denoted by α6 in FIG. 4, whilst the area in the downwards/sidewards direction, viz. the area between the reflected ultrasound beams 7d and 7e, is denoted by α7 in FIG. 4.

The liquid lens 30 enables scanning the ultrasound beam over a certain sector by using the interface between two immiscible liquids, which can be operated by the electrowetting principle. Since the scanning sector is already defined by the liquid lens, the acoustical switch will project it sideward whenever it is desired during treatment. In this case the whole catheter tip should be covered preferably by acoustically transparent material.

The embodiments disclosed in FIGS. 3 and 4 may be combined in order to obtain a continuous scanning along the tip of the catheter. Moreover, instead of using single piston transducer and liquid lens, a phased array transducer (multielement) could be used. This would be especially advantageous when the output acoustical pressure reaches the imaging threshold. In this way the scan sector is provided by the phased array transducer, while its sideward/downwards projection by the acoustical switch and eventually its mechanical operation ensure a large field of view for the catheters.

Further, this might be combined with a liquid lens, whereby three dimensional (3D) imaging is made possible by performing the lateral steering with the array and the elevation steering with the fluid lens. This will result in a large field of view for three dimensional imaging.

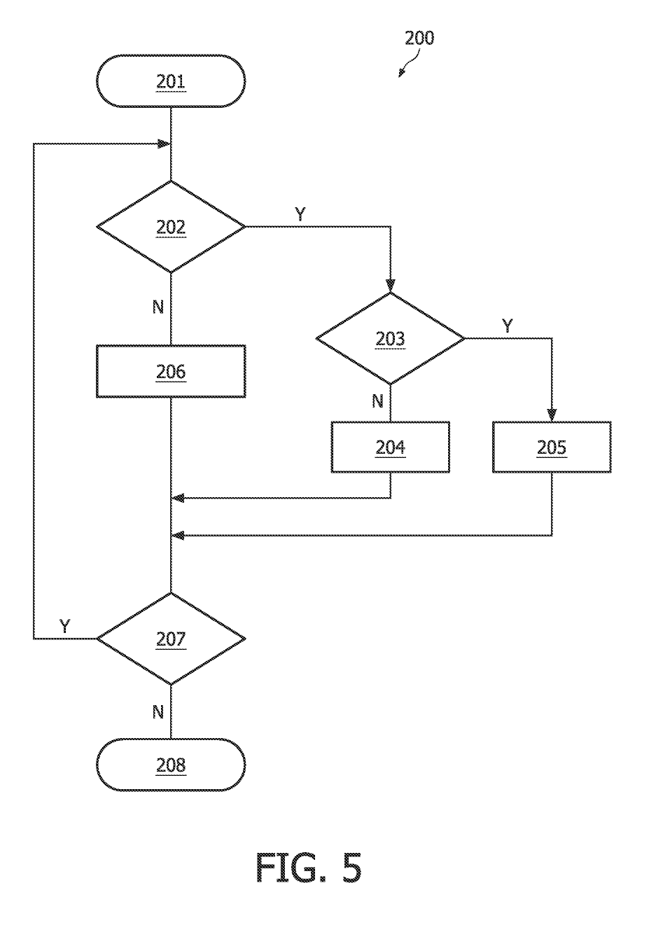

FIG. 5 is a flow-chart of a method 200 of controlling the state of an acoustical switch device for ultrasound according to the invention. The switch device comprises two sheets of acoustically transparent material, where the sheets constitute opposite walls of a housing.

The method 200 starts at 201 and continues to 202, wherein it is decided whether the switch device should be in a transmissive state. In the case, where the decision in step 202 results in the requirement of the switch device to be in a transmissive state, the method continues to step 203, wherein it is decided if the transmissive state is obtained by subjecting the housing of the switch device to underpressure. In the affirmative case, the method continues to step 205, wherein the housing of the switch device is subjected to sufficient underpressure in order to make the sheets of the switch device come into contact with each other, so that no gap exists between them, at least at the part thereof subject to an ultrasound beam from an ultrasound transducer. In the negative case, the method continues from step 203 to step 204, wherein the switch device is made tranmissive by filling the housing thereof with an appropriate liquid, e.g. water. The method continues from either step 205 or step 204 to step 207, wherein it is decided if further or continued control of the switch device is needed. In the affirmative case, the method reverts to step 202; in the negative case the method ends in step 208.

In case where the decision in step 202 is negative, that is the switch device should not be in transmissive state, it should be in a reflective state. In this case, the method continues to step 206, wherein the housing of the switch device is filled with a gas in order to bring the acoustical switch into the desired reflective state. Subsequently, the method continues to step 207, wherein it is decided if further or continued control of the switch device is needed. In the affirmative case, the method reverts to step 202; in the negative case the method ends in step 208.

In short, the invention relates to an acoustical switch wherein the idea to the ultrasound propagation direction may be changes without moving the switch. The switch device comprises two sheets of acoustically transparent material. The sheets constitute opposite walls of a housing. The switch device further comprises one or more orifices for allowing conduction of fluid into and/or out of said housing. The switch device may be made reflective by filling the housing with a gas via the one or more orifices. Moreover, the switch device may be made transmissive to ultrasound by filling the housing with a liquid via the one or more orifices and/or by subjecting the housing to underpressure via the one or more orifices. The acoustical switch device fits well within a catheter, which has severe dimensional limitations.

The switch device of the invention may be used in tissue imaging during treatment, for example treatment of heart arrhythmias. In this case it is desired to follow the progression of lesion formation during the procedure, where the spatial orientation of the catheter changes form perpendicular to parallel with respect to the tissue. Further applications of the switch device of the invention can be in the minimal invasive interventions, where dimensional limitations of the devices are very strict.

Although the present invention has been described in connection with the specified embodiments, it is not intended to be limited to the specific form set forth herein. Rather, the scope of the present invention is limited only by the accompanying claims. In the claims, the term “comprising” does not exclude the presence of other elements or steps.

Additionally, although individual features may be included in different claims, these may possibly be advantageously combined, and the inclusion in different claims does not imply that a combination of features is not feasible and/or advantageous. In addition, singular references do not exclude a plurality. Thus, references to “a”, “an”, “first”, “second” etc. do not preclude a plurality. Furthermore, reference signs in the claims shall not be construed as limiting the scope.

1. An acoustical switch device (10) for ultrasound, wherein said switch device comprises two sheets of acoustically transparent material, said sheets constituting opposite walls (11, 12) of a housing, said switch device further comprising one or more orifices (101) for allowing conduction of fluid into and/or out of said housing.

2. An acoustical switch device (10) according to claim 1, wherein the switch device is made reflective by filling the housing with a gas via the one or more orifices.

3. An acoustical switch device (10) according to claim 1, wherein the switch device is made transmissive to ultrasound by filling the housing with a liquid via the one or more orifices.

4. An acoustical switch device (10) according to claim 1, wherein the switch device is made transmissive to ultrasound by subjecting the housing to underpressure via the one or more orifices.

5. A method (200) of controlling the state of an acoustical switch device for ultrasound, where said switch device comprises two sheets of acoustically transparent material, said sheets constituting opposite walls of a housing, said method comprising the one or more of the following steps:
via the one or more orifices, filling (206) the housing with a gas in order to bring the acoustical switch into a reflective state, and/or
via the one or more orifices, subjecting the housing to underpressure (205) or filling (204) the housing with a liquid in order to bring the acoustical switch into a transmissive state.

6. An ultrasound catheter (100; 110; 120) comprising:
an ultrasound transducer (20); and
a switch device according to claim 1.

7. An ultrasound catheter (100; 110; 120) according to claim 6, further comprising a catheter enclosure accommodating at least part of the switch device and further comprising a liquid, where one or more walls of said catheter enclosure comprising acoustically transparent material.

8. An ultrasound catheter (100; 110; 120) according to claim 6, wherein the ultrasound transducer (20) is a single transducer or an array of transducers.

9. An ultrasound catheter (100; 110; 120) according to claim 6, wherein the transducer (20) is pivotable around an axis angled in relation to an axis of emission of ultrasound from the transducer (20).

10. An ultrasound catheter (100; 110; 120) according to claim 6, wherein the ultrasound transducer further comprises a lens (30) arranged for spreading the ultrasound emitted from the ultrasound transducer.

11. An ultrasound catheter (100; 110; 120) according to claim 10, wherein the lens (30) is a liquid lens operable by the electro wetting principle.

12. An ultrasound catheter (100; 110; 120) according to claim 6, wherein the ultrasound from the ultrasound transducer has an incident angle (α) upon the switch device of about 45°.

13. An ultrasound catheter (100; 110; 120) according to claim 6, further comprising means for actuating the switch device mechanically.

14. A method for directing the ultrasound from an ultrasound catheter comprising an ultrasound transducer and an acoustical switch device according to claim 1, said method comprising:
filling the housing with a gas in order to bring the acoustical switch into a reflective state or subjecting the housing to underpressure or the filling housing with a liquid in order to bring the acoustical switch into a transmissive state, and
subjecting the switch device to ultrasound.

15. A radio frequency ablation catheter (100; 110; 120) comprising an ultrasound transducer and an acoustical switch device according to claim 1.
